(19) United States
(12) Patent Application Publication
(10) Pub. No.: US 2020/0011882 A1
(43) Pub. Date: Jan. 9, 2020

(54) METHOD FOR CALCULATING GLOMERULAR FILTRATION RATE (GFR)

(71) Applicant: TAOYUAN ARMED FORCES GENERAL HOSPITAL, TAOYUAN CITY (TW)

(72) Inventors: PO-JEN HSIAO, NEW TAIPEI CITY (TW); YUN-WEN SHIH, YUNLIN COUNTY (TW); CHI-MING CHU, TAIPEI CITY (TW); SHIH-TAI CHANG, PUZI CITY (TW)

(21) Appl. No.: 16/102,855

(22) Filed: Aug. 14, 2018

(30) Foreign Application Priority Data
Jul. 6, 2018 (TW) 107123574

(51) Int. Cl. G01N 33/70; G01N 33/68; G16H 50/30

(52) U.S. Cl. CPC G01N 33/70; G01N 33/6893; A61B 2090/061; G01N 2800/347; G16H 50/30

(57) ABSTRACT

A method for calculating glomerular filtration rate (GFR) is revealed. A circumference of a patent's neck is measured and then is substituted into an exponential formula together with clinical factors and patient's age for estimating GFR. The present method has a better performance compared with methods for evaluating renal function by GFR available now. The methods available now have poor performance in prediction of loss of renal function at early stage. Some patients are diagnosed at an advanced stage so that they miss the opportunity of early treatment.

### BACKGROUND OF THE INVENTION

### Field of the Invention

The present invention relates to a calculation method, especially to a method for calculating glomerular filtration rate (GFR).

### Description of Related Art

The kidneys are two bean-shaped organs located at the lower back, on two sides of the spine, and within an area between the last (12th) rib and the spine. They are roughly the size of a first and with a weight of about 125-150 gm.

The nephron is the basic unit of the kidney and each kidney contains about 1 million nephrons. Each nephron is composed of the glomerulus and the renal tubule. Blood is filtered as it passes through the nephron and the remaining fluid in the end is urine that is composed of body's waste, water and electrolytes.

Besides removal of metabolic waste and excretion of urine, kidneys contribute to maintain homeostasis, body fluid, electrolytes, and blood pressure. They also secrets erythropoietin. When kidneys are damaged, insufficient erythropoietin is secreted and anemia occurs. Once the kidney function is impaired and unable to remove metabolic waste products, renal failure may happen and patients need to receive hemodialysis (HD) or peritoneal dialysis (PD) that prolongs their life expectancy.

However, many people who like to take medicines always believe this will cure the disease and enhance their health. In fact, the medicines add an extra burden to the liver and kidneys after gastrointestinal absorption of medicines and metabolism of medicines by liver and kidneys.

The dialysis population in Taiwan is expanding at a high rate. Beside drug abuse, the increasing number of patients undergoing dialysis is closely associated with aging population and chronic diseases such as hypertension and diabetes. The average decline in renal function is about 1% per year after age 40.

Not only in Taiwan, increasing attention now is paid to chronic kidney disease (CKD) worldwide because it not only leads to end-stage renal disease (ESRD) but also becomes a major risk factor for cardiovascular diseases and death.

Common high risk factors for Chronic Kidney Disease (CKD) include diabetes mellitus, hypertension, cardiovascular diseases, family history of kidney disease, hyperlipidemia, use of herbal medicines, kidney injury, the elderly, metabolic syndrome, long term use of analgesics, etc.

Thus the renal function should be checked routinely. The glomerular filtration rate (GFR) is defined as the volume of plasma filtered by the glomeruli per unit of time and is an important clinical indicator of kidney function. The normal level of GFR is about 100 ml/min. The methods for estimating GFR available now are as the followings.

(1) Creatinine clearance rate: calculated from the creatinine concentration in a urine sample collected for a consecutive 24-hour period and creatinine concentration in serum. (2) Estimated GFR (eGFR): based on the serum creatinine level and calculated by using Cockcroft-Gault formula and Modification of Diet in Renal Disease (MDRD) formula. (3) Measurement of albuminuria: quantitative analysis and qualitative analysis. The quantitative analysis is carried out by analysis of albumin excreted in a urine sample collected over a 24-hour period. The qualitative analysis is most commonly used and is performed by placing a test paper into urine to read the color change.

Proteinuria is strongly correlated to kidney diseases. The only symptom of a large number of kidney diseases at early stage is proteinuria and the patients feel well. The proteinuria can also be an indicator of kidney caused by some serious systemic diseases (such as diabetes, systemic lupus erythematosus (SLE)).

Albumin is a protein found in blood and there is only a very small amount of albumin in the urine in a normal physical condition (about 20%). When the albuminuria is increased into a moderate level or high level, a pathological condition such as microalbuminuria or macroalbuminuria occurs. The finding of microalbuminuria is the first sign of diabetic kidney disease in clinical practice.

However, the kidney function may be overestimated while being estimated only by creatinine concentration in urine or serum, as revealed in many studies. The serum creatinine may be affected by a plurality of factors including age, gender, race, body size, muscle mass, food, medicine, analytic laboratory methods, etc. Moreover, the formulas available now show poor performance in prediction of loss of renal function at early stage.

Furthermore, the formulas have been developed based on data from Caucasians and African Americans. Thus the results are not as accurate in Asian population and the kidney function is often overestimated. The patients with chronic kidney disease are unable to be screened accurately.

The so-called conventional risk factors for cardiovascular disease development include hypertension, diabetes, dyslipidemia (abnormal lipids), smoking, obesity, etc. These factors also contribute to loss of kidney function and the gradual loss of renal function over time is defined as chronic kidney disease.

The patients with cardiovascular disease are among high risk groups of chronic kidney disease. Along the worse kidney function, the cardiovascular disease also gets worse in order to adapt to changes and maintain homeostasis. For example, the accumulation of water and sodium ions in the body leads to increasing stroke volume, increased activity of peripheral sympathetic nervous system, and increased resistance to peripheral vessels. At last, hypertension occurs. The long term increase in blood pressure and body liquid places a heavy burden on the heart.

Other non-conventional risk factors already known include anemia, metabolic acidosis, an elevated calcium-phosphate (Ca×P) product and hyperparathyroidism. These metabolic abnormalities result in left ventricular hypertrophy and Ventricular Dilation. Thus myocardial remodeling occurs and this leads to heart failure, other cardiovascular diseases, and even death. The patients with chronic kidney disease are also included in high-risk groups of cardiovascular disease.

In addition, studies show that people born with thick neck are easier to have metabolic diseases, sleep apnea, etc. If they have no limits on diet, they have higher risk of cardiovascular problems and diabetes. As a potential risk factor for metabolic syndrome, the neck circumference is better than the waist circumference.

The waist circumference changes with diet (a full or empty stomach) while the neck circumference doesn't. The studies show that the neck circumference and the waist circumference changes are positively correlated in the group of diabetes patients. People whose waist circumference is greater than normal value have the neck circumference higher than normal value.

Most of patients with kidney diseases are not aware of their condition owing to low recognition and high prevalence of chronic kidney disease and no sign of the chronic kidney disease at early stage. The disease reaches at an advanced stage when the symptoms start to appear. This calls for more attention to screening and prevention of the chronic kidney disease.

As to screening for kidney disease, the Cockcroft-Gault formula and the MDRD formula are not applicable to Asian populations. Thus there is room for improvement and there is a need to develop a novel method for estimating glomerular filtration rate (GFR) that is applicable to Asian people.

### SUMMARY OF THE INVENTION

Therefore it is a primary object of the present invention to provide a method for calculating glomerular filtration rate (GFR) in which glomerular filtration rate (GFR) is estimated by endogenous substances in patient's body in combination with patient's neck circumference, age and an exponential formula for assessing renal function now.

In order to achieve the above object, a method for calculating glomerular filtration rate (GFR) according to the present invention includes the following steps. First detect concentration of a plurality of clinical factors in a specimen from a patient to get a plurality of concentration values. The clinical factors include serum creatinine, cystatin C and albuminuria. Then measure a circumference of a patient's neck. Lastly, the concentration values, the circumference, and the patient's age are substituted into an exponential formula to get a glomerular filtration rate (GFR). The exponential formula for estimating the GFR is: 24×(the age)^{-0.495}×(the concentration value of serum creatinine)^{-0.871}×(the concentration value of cystatin C)^{-0.45}×(the circumference)^{0.45}×(the concentration value of albumin in urine)^{0.077}.

When the patient is female, the exponential formula for estimating the GFR is: 24×(the age)^{-0.495}×(the concentration value of serum creatinine)^{-0.871}×(the concentration value of cystatin C)^{-0.45}×(the circumference)^{0.45}×(the concentration value of albumin in urine)^{0.077}×0.502.

A method for calculating glomerular filtration rate (GFR) according to the present invention includes the steps of measuring a circumference of a patient's neck and substituting the circumference and the patient's age into a formula to get a logarithm (log) of glomerular filtration rate (GFR). The formula for calculating the log of GFR is: A+x log (the circumference)+y log (the age) while A is ranging from 0.9 to 4.5, x is ranging from 0.05 to 1.52 and y is ranging from −1.8 to −0.8. The formula for calculating a log of GFR is: A+x log (the circumference)+y log (the age)+B when the patient is female while A is ranging from 0.9 to 4.5, x is ranging from 0.05 to 1.52, y is ranging from −1.8 to −0.8, and B is ranging from −0.1 to 0.1.

A method for calculating glomerular filtration rate (GFR) according to the present invention includes the steps of measuring a circumference of a patient's neck, detecting concentration of a plurality of clinical factors in a specimen from the patient to get a plurality of concentration values, and substituting the concentration values, the circumference, and the patient's age into a formula to get a log of glomerular filtration rate (GFR). The clinical factors include serum creatinine, cystatin C, albuminuria, and a combination thereof.

When the clinical factor is serum creatinine, the formula for calculating a log of GFR is: A+x log (the circumference)+y log (the age)+z log (the serum creatinine) while the formula used to get a log of GFR is: A+x log (the circumference)+y log (the age)+z log (the serum creatinine)+B when the patient is female. In the above formulas, A is ranging from 0.4 to 3.4, x is ranging from 0.2 to 1.5, y is ranging from −1.2 to −0.4, z is ranging from −1.2 to −0.7, and B is ranging from −0.2 to 0.

The formula for calculating a log of GFR is: A+x log (the circumference)+y log (the age)+z log (the serum creatinine)+α log (the albuminuria) when the clinical factors are serum creatinine and albuminuria. The formula for calculating a log of GFR is: A+x log (the circumference)+y log (the age)+z log (the serum creatinine)+α log (the albuminuria)+B when the patient is female. In the above formulas, A is ranging from 0.6 to 3.6, x is ranging from −0.1 to 1.3, y is ranging from −1.1 to −0.3, z is ranging from −1.4 to −0.9, α is ranging from 0 to 0.1 and B is ranging from −0.3 to −0.07.

The formula for calculating a log of GFR is: A+x log (the circumference)+y log (the age)+z log (the serum creatinine)+α log (the albuminuria)+β log (the cystatin C) when the clinical factors are serum creatinine, albuminuria, and cystatin C. When the patient is female, the formula for calculating a log of GFR is: A+x log (the circumference)+y log (the age)+z log (the serum creatinine)+α log (the albuminuria)+β log (the cystatin C)+B. In the above formulas, A is ranging from 0.2 to 3, x is ranging from 0 to 1.3, y is ranging from −0.9 to −1.1, z is ranging from −1.2 to −0.5, α is ranging from 0 to 0.2, β is ranging from −0.8 to −1.1 and B is ranging from −0.2 to 0.

In the step of detecting concentration of a plurality of clinical factors in a specimen from a patient to get a plurality of concentration values, the specimen is selected from the group consisting of blood, and urine.

The urine is collected for a 24-hour period.

In the step of detecting concentration of a plurality of clinical factors in a specimen from a patient to get a plurality of concentration values, the patient is a person with a cardiovascular disease.

The person with a cardiovascular disease can be a patient with heart disease, a patient with blood vessel disorder or a patient with both heart disease and vascular disorder.

In the step of detecting concentration of a plurality of clinical factors in a specimen from a patient to get a plurality of concentration values, the patient is a person with chronic kidney disease.

The patient with chronic kidney disease has kidney damage for more than three months. The kidney damage can be structural damage of kidney, functional damage of kidney or a combination thereof.

The patient with chronic kidney disease is defined as the GFR of less than 60 ml/min/1.73 m² present for more than three months.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a flow chart showing steps of an embodiment according to the present invention;

FIG. 2 is a chart showing correlation of results of an embodiment with glomerular filtration rate (GFR) according to the present invention;

FIG. 3 is a flow chart showing steps of another embodiment according to the present invention;

FIG. 4 is a flow chart showing steps of a further embodiment according to the present invention.

### DETAILED DESCRIPTION OF THE PREFERRED EMBODIMENT

In order to learn features and functions of the present invention, please refer to the following embodiment and related figures.

The most commonly used Cockcroft-Gault or MDRD formula now for estimation of glomerular filtration rate (GFR) is obtained by foreigners' data. The GRF may be underestimated or overestimated in Asian populations. Thereby the present invention provides a method for estimating glomerular filtration rate that overcomes shortcomings of conventional formulas.

The steps and features of the present method for estimating glomerular filtration rate are described in details in the following embodiments.

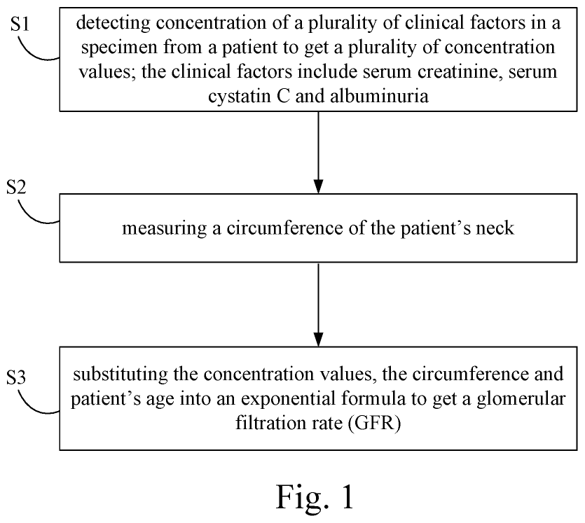

Refer to FIG. 1, a method for calculating glomerular filtration rate (GFR) according to the present invention includes the following steps.

S1: detecting concentration of a plurality of clinical factors in a specimen from a patient to get a plurality of concentration values; the clinical factors include serum creatinine, cystatin C and albuminuria;

S2: measuring a circumference of the patient's neck; and

S3: substituting the concentration values, the circumference and patient's age into an exponential formula to get a glomerular filtration rate (GFR).

As shown in the step S1, a plurality of clinical factors in a specimen from a patient is detected to get a plurality of concentration values. The clinical factors include serum creatinine, cystatin C and albuminuria.

Serum creatinine, cystatin C and albuminuria all can be used to estimate the glomerular filtration rate (GFR). The specimen is selected from the group consisting of blood and urine. The concentration values of serum creatinine and cystatin C are detected in the blood specimens. As to the urine specimen, the concentration of albumin in the urine is detected. The urine is collected over a full 24-hour period.

Serum creatinine refers to creatinine in blood. It's the metabolic creatine phosphate and metabolic creatine in the muscle. The serum creatinine is kept in a stable range and proportional to the muscle mass. A decrease in muscle mass is accompanied by a reduced serum creatinine A part of serum creatinine is filtered and excreted by glomeruli in the kidney.

In patients with urinary elimination problems, the creatinine excretion is affected so that the level of creatinine in the blood rises. This means the renal function is not normal. However, the elderly, patients with muscular dystrophy and bedridden patients may still have normal level of serum creatinine in the blood due to loss of muscle mass. Thus the renal function may be still poor even the level of serum creatinine is normal. The renal function is unable to be assessed only with serum creatinine.

Moreover, cystatin C is filtered freely through glomeruli and then reabsorbed and decomposed by the renal tubular epithelial cells. It doesn't getting back to the blood and couldn't be secreted by renal tubular epithelial cells. The cystatin C serum concentration is mainly determined by glomerular filtration rate (GFR) and less affected by factors such as age, sex, race, muscle mass, etc.

Urine protein excretion in a normal adult should be less than 150 mg per day. Albumin is a kind of protein found in the blood so that a relatively small amount of albumin is lost in the urine. Albumin constitutes approximately 20% of the total urinary protein excreted.

The amount of albumin secreted by the kidneys of normal adults should be below 30 mg (150 mg×20%=30 mg) per day. Normally the kidney doesn't let albumin pass from the blood to the urine. Yet the kidney leaks small amounts of albumin into the urine when pores of the glomerular basement membrane (GBM) are enlarged and large molecules (such as protein) can pass through the glomeruli in large amounts. An albumin level being excreted between 30-300 mg per day is called microalbuminuria while an albumin level above 300 mg per day is called macroalbuminuria.

Microalbuminuria is the earliest clinical indicator of kidney diseases. After proteinuria screening, normal subjects whose urine protein excretion is less than 150 mg per day are further tested whether they have albuminuria or not. The test sample can be a 24-hour urine collection or the first-time urine in the morning. Having inflammation of kidneys, fever or strenuous exercise before the test may affect the result.

As shown in the step S2, measuring a circumference of the patient's neck. The circumference is strongly associated with the carotid intima media thickness. The larger neck circumference means the thicker carotid artery wall. The thicker the carotid artery wall, the smaller the lumen and the greater resistance to blood flow. Thus the blood flow is limited and this leads to metabolic disorders.

The patient has cardiovascular disease that is selected from the group consisting of heart disease, vascular disease and a combination thereof. The cardiovascular diseases mean functional disorders of heart, blood vessels or heart together with blood vessels.

The common cardiovascular diseases include coronary syndrome, stroke, hypertensive heart disease, rheumatic heart disease, cardiomyopathy, congenital heart disease, endocarditis, aortic dissection, vein thrombosis, atherosclerosis, thrombosis, peripheral arterial occlusion disease (PAOD), etc.

Moreover, the patient can be a person with chronic kidney disease that is defined as kidney damage for more than three months. The kidney damage is selected from the group consisting of structural damage of kidney, functional damage of kidney and a combination thereof. The structural damage and functional damage are irreversible, unable to go back to normal. The patient with chronic kidney diseases can also be defined as whose glomerular filtration rate (GFR) is less than 60 ml/min/1.73 m² persistent for more than three months.

The chronic kidney disease is one of the important risk factors that cause cardiovascular diseases. The kidney disease leads to cardiovascular pathology. Similarly, the weakness of cardiovascular function also results in renal progression.

Lastly, as shown in the step S3, the concentration values, the circumference and patient's age are substituted into an exponential formula to get the glomerular filtration rate (GFR).

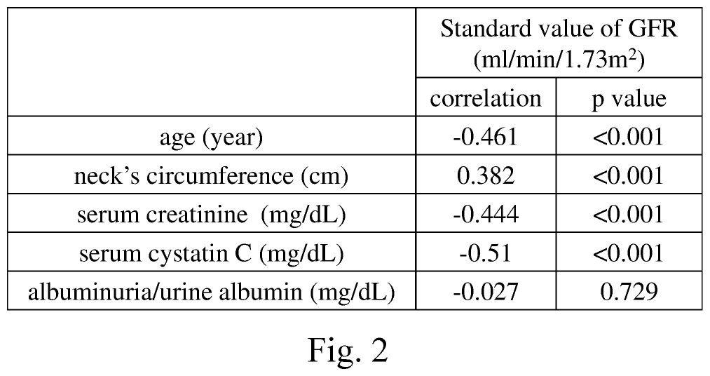

Refer to FIG. 2, results of the above embodiment is revealed. One method of determining normal value of GFR is to collect urine for 24 hours for determination of the creatinine clearance rate (Ccr). The creatinine clearance rate (Ccr) is calculated as: (creatinine concentration in urine (mg/dL)×24-hour urine volume)/(plasma creatinine concentration (mg/dL)×1440 min). Then the Ccr is corrected for the body surface area (BSA) and expressed compared to the average sized man (as 1.73 m²). As shown in the chart, the circumference is positively correlated to the normal value of GFR while serum creatinine, cystatin C and albuminuria are negatively correlated to the normal value of GFR.

The exponential formula for estimating GFR is: 24×(the age)^{-0.495}×(the concentration value of serum creatinine)^{-0.871}×(the concentration value of cystatin C)^{-0.45}×(the circumference)^{0.45}×(the concentration value of albumin in urine)^{0.077}.

Yet the exponential formula for estimating the GFR is: 24×(the age)^{-0.495}×(the concentration value of serum creatinine)^{-0.871}×(the concentration value of cystatin C)^{-0.45}×(the circumference)^{0.45}×(the concentration value of albumin in urine)^{0.077}.×0.502 when the patient is female.

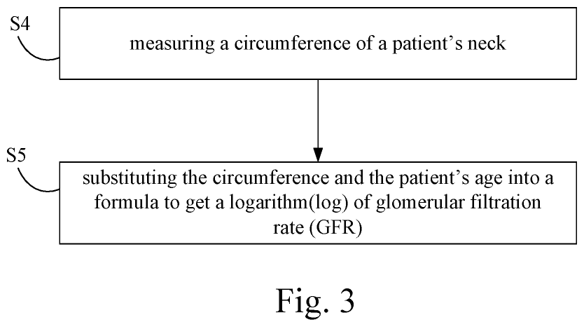

Refer to FIG. 3, another embodiment according to the present invention is disclosed. A method for estimating glomerular filtration rate (GFR) according to the present invention includes the following steps.

S4: measuring a circumference of a patient's neck; and

S5: substituting the circumference and the patient's age into a formula to get a logarithm (log) of glomerular filtration rate (GFR).

The circumference is highly correlated to the carotid intima media thickness. The larger the circumference, the thicker the carotid artery wall and the smaller the lumen. Thus there is the greater resistance to blood flow and metabolic disorders occur as a result of limited blood flow.

The patient has cardiovascular diseases selected from the group consisting of heart diseases, vascular diseases or a combination thereof. The cardiovascular diseases mean functional disorders of heart, blood vessels or both.

The common cardiovascular diseases include coronary syndrome, stroke, hypertensive heart disease, rheumatic heart disease, cardiomyopathy, congenital heart disease, endocarditis, aortic dissection, vein thrombosis, atherosclerosis, thrombosis, peripheral arterial occlusion disease (PAOD), etc.

Moreover, the patient can be a person with chronic kidney disease that is defined as kidney damage present for more than three months. The kidney damage is selected from the group consisting of structural damage of kidney, functional damage of kidney and a combination thereof. The structural damage and functional damage are irreversible, unable to go back to normal. The patient with chronic kidney disease can also be defined as the patient whose glomerular filtration rate (GFR) is less than 60 ml/min/1.73 m² for more than three months.

The chronic kidney disease is one of the important risk factors that cause cardiovascular diseases. The kidney disease leads to cardiovascular pathology. Similarly, the weakness of cardiovascular function also results in renal progression.

The formula for calculating a log of GFR is: A+x log (the circumference)+y log (the age) while A is ranging from 0.9 to 4.5, x is ranging from 0.05 to 1.52 and y is ranging from −1.8 to −0.8. The formula for calculating a log of GFR should be: A+x log (the circumference)+y log (the age)+B when the patient is female while A is ranging from 0.9 to 4.5, x is ranging from 0.05 to 1.52, y is ranging from −1.8 to −0.8, and B is ranging from −0.1 to 0.1.

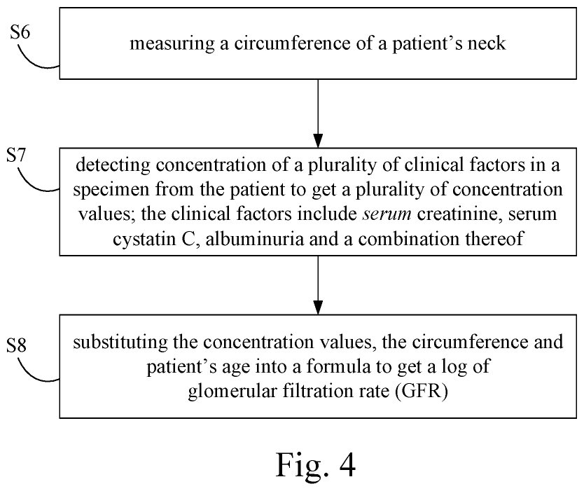

Refer to FIG. 4, a further embodiment is revealed. A method for calculating/estimating glomerular filtration rate (GFR) includes the steps of:

S6: measuring a circumference of a patient's neck;

S7: detecting concentration of a plurality of clinical factors in a specimen from the patient to get a plurality of concentration values; the clinical factors include serum creatinine, cystatin C, albuminuria and a combination thereof; and

S8: substituting the concentration values, the circumference and patient's age into a formula to get a log of glomerular filtration rate (GFR).

When the clinical factor is serum creatinine, the formula for calculating a log of GFR is: A+x log (the circumference)+y log (the age)+z log (the serum creatinine) while A is ranging from 0.4 to 3.4, x is ranging from 0.2 to 1.5, y is ranging from −1.2 to −0.4, and z is ranging from −1.2 to −0.7. The formula used to get a log of GFR is: A+x log (the circumference)+y log (the age)+z log (the serum creatinine)+B when the clinical factor is serum creatinine and the patient is female while A is ranging from 0.4 to 3.4, x is ranging from 0.2 to 1.5, y is ranging from −1.2 to −0.4, z is ranging from −1.2 to −0.7, and B is ranging from −0.2 to 0.

The formula for calculating a log of GFR is: A+x log (the circumference)+y log (the age)+z log (the serum creatinine)+α log (the albuminuria) when the clinical factors are serum creatinine and albuminuria. The formula for calculating a log of GFR is: A+x log (the circumference)+y log (the age)+z log (the serum creatinine)+α log (the albuminuria)+B when the clinical factors are serum creatinine and albuminuria and the patient is female. In the above formulas, A is ranging from 0.6 to 3.6, x is ranging from −0.1 to 1.3, y is ranging from −1.1 to −0.3, z is ranging from −1.4 to −0.9, α is ranging from 0 to 0.1 and B is ranging from −0.3 to −0.07.

The formula for calculating a log of GFR is: A+x log (the circumference)+y log (the age)+z log (the serum creatinine)+α log (the albuminuria)+β log (the cystatin C) when the clinical factors are serum creatinine, albuminuria, and cystatin C while A is ranging from 0.2 to 3, x is ranging from 0 to 1.3, y is ranging from −0.9 to −1.1, z is ranging from −1.2 to −0.5, α is ranging from 0 to 0.2 and β is ranging from −0.8 to −1.1. When the clinical factors are serum creatinine, albuminuria, and cystatin C and the patient is female, the formula for calculating a log of GFR is: A+x log (the circumference)+y log (the age)+z log (the serum creatinine)+α log (the albuminuria)+β log (the cystatin C)+B while A is ranging from 0.2 to 3, x is ranging from 0 to 1.3, y is ranging from −0.9 to −1.1, z is ranging from −1.2 to −0.5, α is ranging from 0 to 0.2, β is ranging from −0.8 to −1.1 and B is ranging from −0.2 to 0.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A method for calculating glomerular filtration rate (GFR) comprising the steps of:
detecting concentration of a plurality of clinical factors in a specimen from a patient to get a plurality of concentration values while the clinical factors having serum creatinine, cystatin C and albuminuria;
measuring a circumference of the patient's neck; and
substituting the concentration values, the circumference and the patient's age into an exponential formula to get a glomerular filtration rate (GFR);
wherein the exponential formula for estimating the GFR is: 24×(the age)^{-0.495}×(the concentration value of serum creatinine)^{-0.871}×(the concentration value of cystatin C)^{-0.45}×(the circumference)^{0.45}×(the concentration value of albuminuria)^{0.077}.

2. The method as claimed in claim 1, wherein the exponential formula for estimating the GFR is: 24×(the age)^{-0.495}×(the concentration value of serum creatinine)^{-0.871}×(the concentration value of cystatin C)^{-0.45}×(the circumference)^{0.45}×(the concentration value of albuminuria)^{0.077}×0.502 when the patient is female.

3. A method for calculating glomerular filtration rate (GFR) comprising the steps of:
measuring a circumference of a patient's neck; and
substituting the circumference and the patient's age into a formula to get a logarithm (log) of glomerular filtration rate (GFR);
wherein the formula for calculating a logarithm (log) of GFR is: A+x log (the circumference)+y log (the age) while A is ranging from 0.9 to 4.5, x is ranging from 0.05 to 1.52 and y is ranging from −1.8 to −0.8; wherein the formula for calculating a logarithm (log) of GFR is: A+x log (the circumference)+y log (the age)+B when the patient is female while A is ranging from 0.9 to 4.5, x is ranging from 0.05 to 1.52, y is ranging from −1.8 to −0.8, and B is ranging from −0.1 to 0.1.

4. A method for calculating glomerular filtration rate (GFR) comprising the steps of:
measuring a circumference of a patient's neck;
detecting concentration of a plurality of clinical factors in a specimen from the patient to get a plurality of concentration values while the clinical factors having serum creatinine, cystatin C, albuminuria and a combination thereof, and
substituting the concentration values, the circumference and the patient's age into a formula to get a logarithm (log) of glomerular filtration rate (GFR).

5. The method as claimed in claim 4, wherein the formula for getting a log of GFR is: A+x log (the circumference)+y log (the age)+z log (the serum creatinine) when the clinical factor is serum creatinine; wherein the formula to get a log of GFR is: A+x log (the circumference)+y log (the age)+z log (the serum creatinine)+B when the clinical factor is serum creatinine and the patient is female; wherein A is ranging from 0.4 to 3.4, x is ranging from 0.2 to 1.5, y is ranging from −1.2 to −0.4, z is ranging from −1.2 to −0.7, and B is ranging from −0.2 to 0.

6. The method as claimed in claim 4, wherein the formula for getting a log of GFR is: A+x log (the circumference)+y log (the age)+z log (the serum creatinine)+α log (the albuminuria) when the clinical factors are serum creatinine and albuminuria; wherein the formula for getting α log of GFR is: A+x log (the circumference)+y log (the age)+z log (the serum creatinine)+α log (the albuminuria)+B when the clinical factors are serum creatinine and albuminuria and the patient is female; wherein A is ranging from 0.6 to 3.6, x is ranging from −0.1 to 1.3, y is ranging from −1.1 to −0.3, z is ranging from −1.4 to −0.9, α is ranging from 0 to 0.1 and B is ranging from −0.3 to −0.07.

7. The method as claimed in claim 4, wherein the formula for getting α log of GFR is: A+x log (the circumference)+y log (the age)+z log (the serum creatinine)+α log (the albuminuria)+β log (the cystatin C) when the clinical factors are serum creatinine, albuminuria, and cystatin C; wherein the formula for getting α log of GFR is: A+x log (the circumference)+y log (the age)+z log (the serum creatinine)+α log (the albuminuria)+β log (the cystatin C)+B when the clinical factors are serum creatinine, albuminuria, and cystatin C and the patient is female; wherein A is ranging from 0.2 to 3, x is ranging from 0 to 1.3, y is ranging from −0.9 to −1.1, z is ranging from −1.2 to −0.5, α is ranging from 0 to 0.2, β is ranging from −0.8 to −1.1 and B is ranging from −0.2 to 0.

8. The method as claimed in claim 1, wherein the specimen is selected from the group consisting of blood and urine in the step of detecting concentration of a plurality of clinical factors in a specimen from a patient to get a plurality of concentration values.

9. The method as claimed in claim 4, wherein the specimen is selected from the group consisting of blood and urine in the step of detecting concentration of a plurality of clinical factors in a specimen from the patient to get a plurality of concentration values.

10. The method as claimed in claim 8, wherein the urine is collected over a 24-hour period.

11. The method as claimed in claim 9, wherein the urine is collected over a 24-hour period.

12. The method as claimed in claim 1, wherein in the step of detecting concentration of a plurality of clinical factors in a specimen from a patient to get a plurality of concentration values, the patient is a person with cardiovascular disease that is selected from the group consisting of heart disease, vascular disease and a combination thereof.

13. The method as claimed in claim 3, wherein the patient is a person with cardiovascular disease that is selected from the group consisting of heart disease, vascular disease and a combination thereof.

14. The method as claimed in claim 4, wherein in the step of detecting concentration of a plurality of clinical factors in a specimen from a patient to get a plurality of concentration values, the patient is a person with cardiovascular disease that is selected from the group consisting of heart disease, vascular disease and a combination thereof.

15. The method as claimed in claim 1, wherein in the step of detecting concentration of a plurality of clinical factors in a specimen from a patient to get a plurality of concentration values, the patient is a person with chronic kidney disease that is defined as kidney damage for more than three months; the kidney damage is selected from the group consisting of structural damage of kidney, functional damage of kidney and a combination thereof.

16. The method as claimed in claim 3, wherein the patient is a person with chronic kidney disease that is defined as kidney damage for more than three months; the kidney damage is selected from the group consisting of structural damage of kidney, functional damage of kidney and a combination thereof.

17. The method as claimed in claim 4, wherein in the step of detecting concentration of a plurality of clinical factors in a specimen from a patient to get a plurality of concentration values, the patient is a person with chronic kidney disease that is defined as kidney damage for more than three months; the kidney damage is selected from the group consisting of structural damage of kidney, functional damage of kidney and a combination thereof.

18. The method as claimed in claim 1, wherein in the step of detecting concentration of a plurality of clinical factors in a specimen from a patient to get a plurality of concentration values, the patient is a person with chronic kidney disease that is defined as the GFR of less than 60 ml/min/1.73 m² for more than 3 months.

19. The method as claimed in claim 3, wherein the patient is a person with chronic kidney disease that is defined as the GFR of less than 60 ml/min/1.73 m² for more than 3 months.

20. The method as claimed in claim 4, wherein in the step of detecting concentration of a plurality of clinical factors in a specimen from a patient to get a plurality of concentration values, the patient is a person with chronic kidney disease that is defined as the GFR of less than 60 ml/min/1.73 m² for more than 3 months.
